(19) United States
(12) Patent Application Publication
(10) Pub. No.: US 2003/0138884 A1
(43) Pub. Date: Jul. 24, 2003

(54) APRIL- a novel protein with growth effects

(75) Inventor: Jurg Tschopp, Epalinges (CH)

Correspondence Address: FISH & NEAVE, 1251 AVENUE OF THE AMERICAS, 50TH FLOOR, NEW YORK, NY, 10020-1105, US

(73) Assignee: Biogen, Inc.

(21) Appl. No.: 10/138,073

(22) Filed: May 1, 2002

Related U.S. Application Data
(62) Division of application No. 09/520,489, filed on Mar. 8, 2000.
Application No. 09/520,489 is a continuation-in-part of application No. PCT/US98/19191, filed on Sep. 11, 1998.
(60) Provisional application No. 60/079,384, filed on Mar. 26, 1998.
(60) Provisional application No. 60/058,786, filed on Sep. 12, 1997.

(51) Int. Cl. C07K 14/705; G01N 33/574; C07H 21/04; C12P 21/02; C12N 5/06

(52) U.S. Cl. 435/69.1; 435/320.1; 435/325; 530/350; 536/23.5

(57) ABSTRACT

APRIL, a novel members of the tumor necrosis factor family (TNF), modified APRILs, and pharmaceutical compositions comprising them.

### RELATED APPLICATIONS

This is a continuation of PCT/US98/19191, filed on Sep. 11, 1998 as a continuation of prior U.S. provisional application Serial No. 60/079,384 filed on Mar. 26, 1998 as a continuation in part of prior U.S. provisional application Serial No. 60/058,786 filed on Sep. 12, 1997.

### FIELD OF THE INVENTION

The present invention relates to novel ligand and polypeptides which are members of the Tumor Necrosis Factor Family. The novel ligand is designated April for “A Proliferation Inducing Ligand.” These proteins or their receptors may have anti-cancer and/or immunoregulatory applications. Furthermore, cells transfected with the genes for these novel ligands may be used in gene therapy to treat tumors, autoimmune and inflammatory diseases or inherited genetic disorders, and blocking antitibodies to these proteins can have immunoregulatory applications.

### BACKGROUND OF THE INVENTION

The tumor-necrosis factor (TNF)-related cytokines are mediators of host defense and immune regulation. Members of this family exist in membrane-anchored forms, acting locally through cell-to-cell contact, or as secreted proteins capable of diffusing to more distant targets. A parallel family of receptors signals the presence of these molecules leading to the initiation of cell death or cellular proliferation and differentiation in the target tissue. Presently, the TNF family of ligands and receptors has at least 13 recognized receptor-ligand pairs, including: TNF:TNF-R; LT-α:TNF-R; LT-α/β:LT-62 -R; FasL:Fas; CD40L:CD40; CD30L:CD30; CD27L:CD27; OX40L:OX40 and 4-1BBL:4-1BB; trance/rankL: Light and Tweak. The DNA sequences encoding these ligands have only about 25% to about 30% identity in even the most related cases, although the amino acid relatedness is about 50%.

The defining feature of this family of cytokine receptors is found in the cysteine rich extracellular domain initially revealed by the molecular cloning of two distinct TNF receptors. (Smith et al., Science, 245, 1019-23 (1990); Kohno et al., PNAS, 87: 8331-5 (1990); Loetscher et al., Cell, 61: 351-9 (1990); Schall et al., Cell, 61: 361-70 (1990). This family of genes encodes glycoproteins characteristic of Type I transmembrane proteins with an extracellular ligand binding domain, a single membrane spanning region and a cytoplasmic region involved in activating cellular functions. The cysteine-rich ligand binding region exhibits a tightly knit disulfide linked core domain, which, depending upon the particular family member, is repeated multiple times. Most receptors have four domains, although there may be as few as three, or as many as six.

Proteins in the TNF family of ligands are characterized by a short N-terminal stretch of normally short hydrophilic amino acids, often containing several lysine or arginine residues thought to serve as stop transfer sequences. Next follows a transmembrane region and an extracellular region of variable length, that separates the C-terminal receptor binding domain from the membrane. This region is sometimes referred to as the “stalk”. The C-terminal binding region comprises the bulk of the protein, and often, but not always, contains glycosylation sites. These genes lack the classic signal sequences characteristic of type I membrane proteins, type II membrane proteins with the C terminal domain lying outside the cell, and a short N-terminus residing in the cytoplasm. In some cases, e.g., TNF and LT-α, cleavage in the stalk region can occur early during protein processing and the ligand is then found primarily in secreted form. Most ligands, however, exist in a membrane form, mediating localized signaling.

The structure of these ligands has been well-defined by crystallographic analyses of TNF, LT-α, and CD40L. TNF and lymphotoxin-α (LT-α) are both structured into a sandwich of two anti-parallel β-pleated sheets with the “jelly roll” or Greek key topology. (See Jones et al., Nature, 338, 225-8 (1989); Eck et al., J. Biol. Chem., 264: 17595-605 (1992). The rms deviation between the Cα and β residues is 0.61 C, suggesting a high degree of similarity in their molecular topography. A structural feature emerging from molecular studies of CD40L, TNF and LT-α is the propensity to assemble into oligomeric complexes. Intrinsic to the oligomeric structure is the formation of the receptor binding site at the junction between the neighboring subunits creating a multivalent ligand. The quaternary structures of TNF, CD40L and LT-α have been shown to exist as trimers by analysis of their crystal structures. Many of the amino acids conserved between the different ligands are in stretches of the scaffold β-sheet. It is likely that the basic sandwich structure is preserved in all of these molecules, since portions of these scaffold sequences are conserved across the various family members. The quaternary structure may also be maintained since the subunit conformation is likely to remain similar.

TNF family members can best be described as master switches in the immune system controlling both cell survival and differentiation. Only TNF and LTα are currently recognized as secreted cytokines contrasting with the other predominantly membrane anchored members of the TNF family. While a membrane form of TNF has been well-characterized and is likely to have unique biological roles, secreted TNF functions as a general alarm signaling to cells more distant from the site of the triggering event. Thus TNF secretion can amplify an event leading to the well-described changes in the vasculature lining and the inflammatory state of cells. In contrast, the membrane bound members of the family send signals though the TNF type receptors only to cells in direct contact. For example T cells provide CD40 mediated “help” only to those B cells brought into direct contact via cognate TCR interactions. Similar cell-cell contact limitations on the ability to induce cell death apply to the well-studied Fas system.

It appears that one can segregate the TNF ligands into three groups based on their ability to induce cell death (Table III). First, TNF, Fas ligand and TRAIL can efficiently induce cell death in many lines and their receptors mostly likely have good canonical death domains. Presumably the ligand to DR-3 (TRAMP/WSL-1) would also all into this category. Next there are those ligands which trigger a weaker death signal limited to few cell types and TWEAK, CD30 ligand and LTIαβ2 are examples of this class. How this group can trigger cell death in the absence of a canonical death domain is an interesting question and suggests that a separate weaker death signaling mechanism exists. Lastly, there are those members that cannot efficiently deliver a death signal. Probably all groups can have antiproliferative effects on some cell types consequent to inducing cell differentiation e.g. CD40 (Funakoshi et al., 1994).

The TNF family has grown dramatically in recent years to encompass at least 11 different signaling pathways involving regulation of the immune system. The widespread expression patterns of TWEAK and TRAIL indicate that there is still more functional variety to be uncovered in this family. This aspect has been especially highlighted recently in the discovery of two receptors that affect the ability of rous sarcoma and herpes simplex virus to replicate as well as the historical observations that TNF has anti-viral activity and pox viruses encode for decoy TNF receptors (Brojatsch et al., 1996; Montgomery et al., 1996; Smith, 1994; Vassalli, 1992). TNF is a mediator of septic shock and cachexia (K. Tracey, in Tumor Necrosis Factors. The Molecules and Their Emerging Role in Medicine, B. Beutler (Ed.), Raven Press, NY, p 255 (1992)); A. Waage, in Tumor Necrosis Factors. The Molecules and Their Emerging Role in Medicine, B. Beutler (Ed.), Raven Press, NY, p 275 (1992), and is involved in the regulation of hematopoietic cell development. (G. D. Roodman, in Tumor Necrosis Factors. The Molecules and Their Emerging Role in Medicine, B. Beutler (Ed.), Raven Press, NY, p 117 (1992). It appears to play a major role as a mediator of inflammation and defense against bacterial, viral and parasitic infections (A. Nakane, in Tumor Necrosis Factors. The Molecules and Their Emerging Role in Medicine, B. Beutler (Ed.), Raven Press, NY, p 285 (1992); I. A. Clark et al., in Tumor Necrosis Factors. The Molecules and Their Emerging Role in Medicine, B. Beutler (Ed.), Raven Press, NY, p 303 (1992); G. E. Grau et al., in Tumor Necrosis Factors. The Molecules and Their Emerging Role in Medicine, B. Beutler (Ed.), Raven Press, NY, p 329 (1992); P-F. Piguet, in Tumor Necrosis Factors. The Molecules and Their Emerging Role in Medicine, B. Beutler (Ed.), Raven Press, NY, p 341 (1992); G. H. Wong et al., in Tumor Necrosis Factors. The Molecules and Their Emerging Role in Medicine, B. Beutler (Ed.), Raven Press, NY, p 371 (1992) as well as having antitumor activity. (S. Malik, in Tumor Necrosis Factors. The Molecules and Their Emerging Role in Medicine, B. Beutler (Ed.), Raven Press, NY, p 407 (1992). TNF is also involved in different autoimmune diseases. (D. A. Fox, Am. J. Med., 99, p 82 (1995). TNF may be produced by several types of cells, including macrophages, fibroblasts, T cells and natural killer cells. (D. Goeddel et al., Cold Spring Harbor Symposium Quant. Biol., 51, p 597 (1986); G. Trinchieri, in Tumor Necrosis Factors. The Molecules and Their Emerging Role in Medicine, B. Beutler (Ed.), Raven Press, NY, p 515 (1992). TNF binds to two different receptors, each -acting through. specific intracellular signaling molecules, thus resulting in different effects of TNF. (L. A. Tartaglia et al., Proc. Natl. Acad. Sci. USA, 88, p 9292 (1991); L. A. Tartaglia and D. V. Goeddel, Immunol. Today, 13, p 151 (1992). TNF can exist either as a membrane bound form or as a soluble secreted cytokine. (B. Luettig et al., J. Immunol., 143, p 4034 (1989); M. Kriegler et al., Cell, 53, p 45 (1988).

LT-α shares many activities with TNF, i.e. binding to the TNF receptors, (C. F. Ware et al., in Pathways for Cytolysis, G. M. Griffiths and J. Tschopp (Eds.), Springer-Verlag, Berlin, Heidelberg, p175-218 (1995), but unlike TNF, appears to be secreted primarily by activated T cells and some β-lymphoblastoid tumors. (N. Paul et al., Ann. Rev. Immunol., 6, p 407 (1988). The heteromeric complex of LT-α and LT-β is a membrane bound complex which binds to the LT-β receptor. (P. D. Crowe et al., Science, 264, p 707 (1994). (J. Browning et al., Cell, 72, p 847 (1993); J. Browning et al., J. Immunol., 154, p 33 (1995). The LT system (LTs and LT-R) appears to be involved in the development of peripheral lymphoid organs since genetic disruption of LT-β leads to disorganization of T and B cells in the spleen and an absence of lymph nodes. (P. De Togni et al., Science, 264, p 703 (1993); T. A. Banks et al., J. Immunol., 155, p 1685 (1995).

The LT-β system is also involved in cell death of some adenocarcinoma cell lines. (J. Browning and A. Ribolini, J. Immunol., 143, p1859 (1989): J. Browning et al., J. Exp. Med., 183, p 867 (1996).

Fas-L, another member of the TNF family, is expressed predominantly on activated T cells. (T. Suda et al., J. Immunol., 154, p 3806 (1995); (T. Suda et al., J. Immunol., 154, p 3806 (1995). It induces the death of cells bearing its receptor, including tumor cells and HIV-infected cells, by a mechanism known as programmed cell death or apoptosis. (B.C. Trauth et al., Science, 245, p 301 (1989); S. Yonehara et al., J. Exp. Med., 169, p1747 (1989); S. Nagata and P. Goldstein, Science, 267, p 1449 (1995); M. H. Falk et al., Blood, 79, p 3300 (1992). Furthermore, deficiencies in either Fas or Fas-L may lead to lymphoproliferative disorders, confirming the role of the Fas system in the regulation of immune responses. (F. Rieux-Laucat et al., Science, 268, p1347 (1995); T. Takahashi et al., Cell, 76, p 969 (1994); R. Watanabe-Fukunaga et al., Nature, 356, p 314 (1992). The Fas system is also involved in liver damage resulting from hepatitis chronic infection (P. R. Galle and al., J. Exp. Med., 182, p 1223 (1995) and in autoimmunity in HIV-infected patients. (F. Silvestris and al., Clin. Immunol. Immunopathol., 75, p 197 (1995). The Fas system is also involved in T-cell destruction in HIV patients. (P. D. Katsikis et al., J. Exp. Med., 181, p 2029 (1995); A. D. Badley et al., J. Virol., 70, p 199 (1996). TRAIL, another member of this family, also seems to be involved in the death of a wide variety of transformed cell lines of diverse origin. (S. Wiley et al., Immunity, 3, p 673 (1995).

CD40-L, another member of the TNF family, is expressed on T cells and induces the regulation of CD40-bearing B cells. (J. F. Gauchat et al., FEBS Lett., 315, p 259 (1993); S. Funakoshi et al., Blood, 83, p 2787 (1994). Furthermore, alterations in the CD40-L gene result in a disease known as X-linked hyper-IgM syndrome. (R. C.

Allen et al., Science, 259, p 990 (1993). The CD40 system is also involved in different autoimmune diseases (L. Biancone et al., Kidney-Int., 48, p 458 (1995); C. Mohan et al., J. Immunol., 154, p 1470 (1995) and CD40-L is known to have antiviral properties. (J. Ruby and al., Nature Medicine, 1, p 437 (1995). Although the CD40 system is involved in the rescue of apoptotic B cells, (Z. Wang et al., J. Immunol., 155, p 3722 (1995); A. M. Cleary and al., J. Immunol., 155, p 3329 (1995) in non-immune cells it induces apoptosis. (S. Hess and H. Engelman, J. Exp. Med., 183, p 159 (1996). Many additional lymphocyte members of the TNF family are also involved in costimulation. (R. G. Goodwin et al, Cell, 73, p 447 (1993); Goodwin et al, Eur. J. Immunol., 23, p 2631 (1993); C. A. Smith et al., Cell, 73, p 1349 (1993).

Generally, the members of the TNF family have fundamental regulatory roles in controlling the immune system and activating acute host defense systems. Given the current progress in manipulating members of the TNF family for therapeutic benefit, it is likely that members of this family may provide unique means to control disease. Some of the ligands of this family can directly induce the apoptotic death of many transformed cells e.g. LT, TNF, Fas ligand and TRAIL (Nagata, 1997). Fas and possibly TNF and CD30 receptor activation can induce cell death in nontransformed lymphocytes which may play an immunoregulatory function (Amakawa et al., 1996; Nagata, 1997; Sytwu et al., 1996; Zheng et al., 1995). In general, death is triggered following the aggregation of death domains which reside on the cytoplasmic side of the TNF receptors. The death domain orchestrates the assembly of various signal transduction components which result in the activation of the caspase cascade (Nagata, 1997). Some receptors lack canonical death domains, e.g. LTb receptor and CD30 (Browning et al., 1996; Lee et al., 1996) yet can induce cell death, albeit more weakly. It is likely that these receptors function primarily to induce cell differentiation and the death is an aberrant consequence in some transformed cell lines, although this picture is unclear as studies on the CD30 null mouse suggest a death role in negative selection in the thymus (Amakawa-et al., 1996). Conversely, signaling through other pathways such as CD40 is required to maintain cell survival. Thus, there is a need to identify and characterize additional molecules which are members of the TNF family thereby providing additional means of controlling disease and manipulating the immune system.

It has been suggested that certain members of the TNF family may provide therapeutic anti-tumor benefits, for example, in combination with IL-2. (See, e.g. U.S. Pat. No. 5,425,940). However, to date, no completely satisfactory treatment for cancer is known. Combination chemotherapy is commonly used in the clinic and in research, for example with antimetabolites, alkylating agents, antibiotics, general poisons, etc. Such drugs are administered alone or in combination in an attempt to obtain a cytotoxic effect on cancers, and/or to reduce or eliminate the emergence of drug-resistant cells, and to reduce side effects.

### SUMMARY OF THE INVENTION

Accordingly, the present invention is directed to a novel polypeptide referred to as APRIL, which substantially obviates one or more of the problems due to the limitations and disadvantages of the related art. The inventors have discovered a new member of the TNF family of cytokines, and defined both the human and murine amino acid sequence of the protein, as well as the DNA sequences encoding these proteins. The claimed invention may be used to identify new diagnostics and therapeutics for numerous diseases and conditions as discussed in more detail below, as well as to obtain information about, and manipulate, the immune system and its processes. Additionally, the invention may be involved in the induction of cell death in carcinomas.

Additional features and advantages of the invention will be set forth in the description which follows, and in part will be apparent from the description, or may be learned by practice of the invention. The objectives and other advantages of the invention will be realized and attained by the compositions and methods particularly pointed out in the written description and claims hereof, as well as in the appended drawings.

Thus, to achieve these and other advantages, and in accordance with the purpose of the invention, as embodied and broadly described herein, the invention includes DNA sequences encoding APRIL. Specifically, the invention relates to DNA sequences which encode human APRIL, (SEQ. ID. NO. 1). Additionally, the claimed invention relates to the amino acid sequences of this novel ligand. The amino acid sequence of human APRIL is set forth in SEQ. ID. NO.: 2. Additionally, the inventors have set forth herein the DNA and amino acid sequences for murine APRIL, set forth in SEQ. ID. NOS. 3 and 4 respectively. In other embodiments, the invention relates to sequences that have at least 50% homology with DNA sequences encoding the C terminal receptor binding domain of this ligand and when hybridize to the claimed DNA sequences or fragments thereof, and which encode APRIL having the sequence identified in SEQ. ID. NO. 1 or a protein having similar biological activity.

The invention in certain embodiments furthermore relates to DNA sequences encoding APRIL where the sequences are operatively linked to an expression control 5 sequence. Any suitable expression control sequences are useful in the claimed invention, and can easily be selected by one skilled in the art.

The invention also contemplates recombinant DNAs comprising a sequence encoding APRIL or fragments thereof, as well as hosts with stably integrated APRIL sequences introduced into their genome, or possessing episomal elements. Any suitable host may be used in the invention, and can easily be selected by one skilled in the art without undue experimentation.

In other embodiments, the invention relates to methods of producing substantially pure APRILs comprising the step of culturing transformed hosts. In yet other embodiments, the invention relates to APRIL essentially free of normally associated animal proteins.

The invention encompasses APRIL ligands having the amino acid sequence identified in SEQ. ID. NO. 2, as well as fragments or homologs thereof. In various embodiments, the amino acid and/or the DNA sequences may comprise conservative insertions, deletions and substitutions, as further defined below or may comprise fragments of said sequences.

The invention relates in other embodiments to soluble constructs comprising APRIL, which may be used to directly trigger APRIL mediated pharmacological events. Such events may have useful therapeutic benefits in the stimulation of growth, treatment of cancer, tumors or the manipulation of the immune system to treat immunologic diseases. Soluble forms of the claimed ligands could be genetically reengineered to incorporate an easily recognizable tag, thereby facilitating the identification of the receptors for these ligands.

Additionally, certain embodiments relate to antibodies against APRIL, and their use for the treatment of cancers, tumors, or manipulation of the immune system to treat immunologic diseases.

In yet other embodiments the invention relates to methods of gene therapy using the genes for APRIL as disclosed and claimed herein.

The pharmaceutical preparations of the invention may, optionally, include pharmaceutically acceptable carriers, adjuvants, fillers, or other pharmaceutical compositions, and may be administered in any of the numerous forms or routes known in the art.

It is to be understood that both the foregoing general description and the following detailed description are exemplary and explanatory, and are intended to provide further explanation of the invention as claimed.

The accompanying drawings are included to provide a further understanding of the invention, and are incorporated in, and constitute a part of this specification, illustrate several embodiments of the invention, and together with the description serve to explain the principles of the invention.

### BRIEF DESCRIPTION OF THE DRAWINGS

Figure Legends

FIG. 1. (A) Predicted amino acid sequence of human APRIL. The predicted transmembrane region (TM, boxed), the potential N-linked glycosylation site (star) and the N-terminus of the recombinant soluble APRIL (sAPRIL) are indicated. (B) Comparison of the extracellular protein sequence of APRIL and some members of the TNF ligand family. Identical and homologuous residues are represented in black and shaded boxes, respectively. TNFa, tumor necrosis factor α, LTa, (lymphotoxin α), FasL, (Fas (CD95) ligand), TRAIL, TWEAK and TRANCE, (RANK ligand).

FIG. 2. Expression of APRIL (A) Northern blots (2 μg poly A⁺ RNA per lane) of various human tissues were probed with APRIL cDNA. (B) APRIL mRNA expression in various tumor cell lines: promyelocitic leukemia HL 60; HeLa Cell S3; chronic myelogenous leukemia K562; lymphoblastic leukemia Molt4; Burkitt's lymphoma Raji; colorectal adenocarcinoma A459; melanoma G361. (C) APRIL mRNA expression in four different human tumors (T) and normal tissues (N). The 18S rRNA band shows equal loading. (D) APRIL mRNA expression in primary colon carcinoma. In situ hybridization revealed abundant APRIL message in human colon carcinoma as compared to normal colon tissue. Colon tumor tissue sections and adjacent normal colon tissue were hybridized to antisense APRIL ³⁵S-labeled cRNA, and as control, colon tumor tissue sections were also hybridized to sense APRIL ³⁵S cRNA (negative control). The upper panels are dark field micrographs, the lower panels are the corresponding light field micrographs.

FIG. 3. APRIL stimulates cell growth. (A) Dose dependent increase of proliferation of Jurkat (human leukemia T cells), as determined 24 hrs after addition of soluble APRIL. Controls are Fas ligand (FasL), TWEAK and no ligand (Control) (left panel, cell viability; right panel, ³H-Thymidine incorperation). (B) Influence of immunodepletion of FLAG-tagged APRIL on tumor cell growth. The proliferative effect of FLAG-tagged APRIL is neutralized by anti-FLAG antibodies, but not by anti-myc antibodies. (C) Effect of APRIL on the proliferation rate of Raji (human Burkitt lymphoma B cells), A20 cells (mouse B lymphoma), BJAB (human B lymphoma), COS (canine epithelial cells), MCF-7 (human breast adenocarcinoma), HeLa (human epitheloid carcinoma) and ME260 (human melanoma). (1)) Influence of fetal calf serum concentration on APRIL-induced proliferation of Jurkat cells.

FIG. 4. APRIL accelerates tumor growth. (A) Characterization of APRIL-transfected NIH-3T3 clones. FLAG-APRIL levels of the various clones were analyzed by Western blotting using an anti-FLAG antibody. The arrow points to the APRIL protein, the high molecular weight protein is detected non-specifically (B) APRIL-expressing NIH-3T3 clones grow faster than mock-transfeced clones. (C) Increased tumor growth of APRIL-expressing NIH-3T3 clones. NIH-3T3 cells (1×10⁵ cells) and APRIL (NIH-AP, 2 different clones) transfectants (1×10⁶ cells) were injected subcutaneously into nude mice, and tumor growth monitored.

FIG. 5. An alignment of the human and mouse APRIL amino acid sequences showing the extensive identity between the two proteins. Identical residues are marked with the overlaying dot. The underlined residues represent a potential N-linked glycosylation site. The intiating methionine is considered a likely start site, however, it is possible that in frame methionines further upstream may serve as the actual start site, for example, in the human sequence.

### DETAILED DESCRIPTION

Reference will now be made in detail to the present preferred embodiments of the invention. This invention relates to DNA sequences that code for human or mouse APRIL, fragments and homologs thereof, and expression of those DNA sequences in hosts transformed with them. The invention relates to uses of these DNA sequences and the peptides encoded by them. Additionally, the invention encompasses both human and mouse amino acid sequences for and APRIL, or fragments thereof, as well as pharmaceutical compositions comprising or derived from them. The invention relates to methods of stimulating cell growth with APRIL, or, alternatively, methods of inhibiting tumorogenesis using antibodies directed against APRIL or a receptor of APRIL.

A. DEFINITIONS

“Homologous”, as used herein, refers to the sequence similarity between sequences of molecules being compared. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

As used herein, the term “cancer” refers to any neoplastic disorder, including such cellular disorders as, for example, renal cell cancer, Kaposi's sarcoma, chronic leukemia, breast cancer, sarcoma, ovarian carcinoma, rectal cancer, throat cancer, melanoma, colon cancer, bladder cancer, mastocytoma, lung cancer, mammary adenocarcinoma, pharyngeal squamous cell carcinoma, and gastrointestinal or stomach cancer. Preferably, the cancer is leukemia, mastocytoma, melanoma, lymphoma, mammary adenocarcinoma, and pharyngeal squamous cell carcinoma

A “purified preparation” or a “substantially pure preparation” of a polypeptide, as used herein, means a polypeptide that has been separated from other proteins, lipids, and nucleic acids with which it naturally occurs. Preferably, the polypeptide is also separated from other substances, e.g., antibodies, matrices, etc., which are used to purify it. “Transformed host” as used herein is meant to encompass any host with stably integrated sequence, i.e. a sequence encoding APRIL, introduced into its genome.

A “treatment”, as used herein, includes any therapeutic treatment, e.g., the administration of a therapeutic agent or substance. e.g., a drug.

A “substantially pure nucleic acid”, e.g., a substantially pure DNA, is a nucleic acid which is one or both of: (1) not immediately contiguous with either one or both of the sequences, e.g., coding sequences, with which it is immediately contiguous (i.e., one at the 5′ end and one at the 3′ end) in the naturally-occurring genome of the organism from which the nucleic acid is derived; or (2) which is substantially free of a nucleic acid sequence with which it occurs in the organism from which the nucleic acid is derived. The term includes, for example, a recombinant DNA which is incorporated into a vector, e.g., into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment) independent of other DNA sequences. Substantially pure DNA also includes a recombinant DNA which is part of a hybrid gene encoding APRIL.

The terms “peptides”, “proteins”, and “polypeptides” are used interchangeably herein.

“Biologically active” as used herein, means having an in vivo or in vitro activity which may be performed directly or indirectly. Biologically active fragments of APRIL may have, for example, 70% amino acid homology with the active site of APRIL, more preferably at least 80%, and most preferably, at least 90% homology. Identity or homology with respect to APRIL is defined herein as the percentage of amino acid residues in the candidate sequence which are identical to the APRIL residues in SEQ. ID. NOS. 2 or 4.

“Ligand” as used herein generically refers to APRIL. The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature.

Introduction:

APRIL, a novel member of the TNF family, is described in detail herein. The inventors have found that while transcript of APRIL are of low abundance in normal tissues, high levels of mRNA are detected in several tumor cell lines, as well as in colon carcinomas, metastatic lymphomas and thyroid tumors. In vitro, the addition of recombinant APRIL stimulates the proliferation of various cell lines. Moreover, transfection of APRIL into NIH-3T3 cells dramatically accelerated tumor growth in nude mice when compared to mock transfectants. The expression and growth stimulating effect of APRIL on tumor cells in vitro and in vivo suggests that APRIL is implicated in tumorigenesis.

APRIL appears to be unique among the members of the TNF family as it is both abundantly expressed in tumor cells and stimulates growth of many different tumor cell lines given the apparent role of APRIL is tumorigenesis, the antagonistic antibodies to APRIL, or the APRIL receptor, will provide novel approaches to cancer treatment.

B. DNA SEQUENCES OF THE INVENTION

As described herein, one aspect of the invention features a substantially pure (or recombinant) nucleic acid which includes a nucleotide sequence encoding APRIL, such as the DNA described in SEQ. ID. NO. 1 and/or equivalents of such nucleic acids. The term nucleic acid as used herein can include fragments and equivalents, such as, for example, sequences encoding functionally equivalent peptides. Equivalent nucleotide sequences may include sequences that differ by one or more nucleotide substitutions, additions or deletions, such as allelic variants, mutations, etc. and include sequences that differ from the nucleotide sequence encoding APRIL shown in SEQ. ID NO: 1 due to the degeneracy of the genetic code.

The invention will be described generally by reference to the human sequences, although one skilled in the art will understand that the mouse sequences or sequences encoding APRIL from other species having a high level of homology with human, and are encompassed herein. The human proteins appear to have all of the characteristics of the TNF family, i.e., a type II membrane protein organization and conservation of the sequence motifs involved in the folding of the protein into the TNF anti-parallel θ sheet structure.

The sequences of the invention can be used to prepare a series of DNA probes that are useful in screening various collections of natural and synthetic DNAs for the presence of DNA sequences that are closely related to APRIL, or fragments or derivatives thereof. One skilled in the art will recognize that reference to APRIL as used herein, refers also to biologically active derivatives, fragments or homologs thereof.

The DNA sequences of the invention coding on APRIL can be employed to produce the claimed peptides on expression in various prokaryotic and eukaryotic hosts transformed with them. These peptides may be used in anti-cancer, and immunoregulatory applications. In general, this comprises the steps of culturing a host transformed with a DNA molecule containing the sequence encoding APRIL, operatively-linked to an expression control sequence.

The DNA sequences and recombinant DNA molecules of the present invention can be expressed using a wide variety of host/vector combinations. For example, useful vectors may consist of segments of chromosomal, non-chromosomal or synthetic DNA sequences. The expression vectors of the invention are characterized by at least one expression control sequence that may be operatively linked to the APRIL DNA sequence inserted in the vector, in order to control and to regulate the expression of the DNA sequence.

Furthermore, within each expression vector, various sites may be selected for insertion of a sequence of the invention. The sites are usually designated by a restriction endonuclease which cuts them, and these sites and endonucleases are well recognized by those skilled in the art It is of course to be understood that an expression vector useful in this invention need not have a restriction endonuclease site for insertion of the desired DNA fragment. Instead, the vector may be cloned to the fragment by alternate means. The expression vector, and in particular the site chosen therein for insertion of a selected DNA fragment, and its operative linking therein to an expression control sequence, is determined by a variety of factors. These factors include, but are not limited to, the size of the protein to be expressed, the susceptibility of the desired protein to proteolytic degradation by host cell enzymes, number of sites susceptible to a particular restriction enzyme, contamination or binding of the protein to be expressed by host cell proteins which may prove difficult to remove during purification. Additional factors which may be considered include expression characteristics such as the location of start and stop codons relative to the vector sequences, and other factors which will be recognized by those skilled in the art. The choice of a vector and insertion site for the claimed DNA sequences is determined by a balancing of these factors, not all selections being equally effective for a desired application. However, it is routine for one skilled in the art to analyze these parameters and choose an appropriate system depending on the particular application.

One skilled in the art can readily make appropriate modifications to the expression control sequences to obtain higher levels of protein expression, i.e. by substitution of codons, or selecting codons for particular amino acids that are preferentially used by particular organisms, to minimize proteolysis or to alter glycosylation composition. Likewise, cysteines may be changed to other amino acids to simplify production, refolding or stability problems.

Thus, not all host/expression vector combinations function with equal efficiency in expressing the DNA sequences of this invention. However, a particular selection of a host/expression vector combination may be made by those of skill in the art. Factors one may consider include, for example, the compatibility of the host and vector, toxicity to the host of the proteins encoded by the DNA sequence, ease of recovery of the desired protein, expression characteristics of the DNA sequences and expression control sequences operatively linked to them, biosafety, costs and the folding, form or other necessary post-expression modifications of the desired protein.

APRIL produced by hosts transformed with the sequences of the invention, as well as native APRIL purified by the processes of this invention, or produced from the claimed amino acid sequences, are useful in a variety of compositions and methods for anticancer, antitumor and immunoregulatory applications. They are also useful in therapy and methods directed to other diseases.

This invention also relates to the use of the DNA sequences disclosed herein to express APRIL under abnormal conditions, i.e. in a gene therapy setting. Additionally, APRIL may be expressed in tumor cells under the direction of promoters appropriate for such applications. Such expression could enhance anti-tumor immune responses or directly affect the survival of the tumor. APRIL is also likely to affect the survival of an organ graft by altering the local immune response. In this case, the graft itself or the surrounding cells would be modified with an engineered gene encoding APRIL.

Another aspect of the invention relates to the use of the isolated nucleic acid encoding either APRIL in “antigens” therapy. As used herein, “antisense” therapy refers to administration or in situ generation of oligonucleotides or their derivatives which specifically hybridize under cellular conditions with the cellular mRNA and/or DNA encoding the APRIL sequence of interest, so as to inhibit expression of the encoded protein, i.e. by inhibiting transcription and/or translation. The binding may be by conventional base pair complementarity, or, for example, in the case of binding to DNA duplexes, through specific interactions in the major groove of the double helix. In general, “antisense” therapy refers to a range of techniques generally employed in the art, and includes any therapy which relies on specific binding to oligonucleotide sequences.

An antisense construct of the present invention can be delivered, for example, as an expression plasmid, which, when transcribed in the cell, produces RNA which is complementary to at least a portion of the cellular mRNA which encodes APRIL. Alternatively, the antisense construct can be an oligonucleotide probe which is generated ex vivo. Such oligonucleotide probes are preferably modified oligonucleotides which are resistant to endogenous nucleases, and are therefor stable in vivo. Exemplary nucleic acids molecules for use as antisense oligonucleotides are phosphoramidates, phosphothioate and methylphosphonate analogs of DNA (See, e.g., U.S Pat. Nos. 5,176,996; 5,264,564; and 5,256,775). Additionally, general approaches to constructing oligomers useful in antisense therapy have been reviewed, for example, by Van Der Krol et al., (1988) Biotechniques 6:958-976; and Stein et al. (1988) Cancer Res 48: 2659-2668, specifically incorporated herein by reference.

C. APRIL AND AMINO ACID SEQUENCES THEREFOR

APRIL, as discussed above, is a member of the TNF family. The protein, fragments or homologs of APRIL may have wide therapeutic and diagnostic applications as discussed in more detail below.

Although the precise three dimensional structure of APRIL is not known, it is predicted that, as a member of the TNF family, it may share certain structural characteristics with other members of the family.

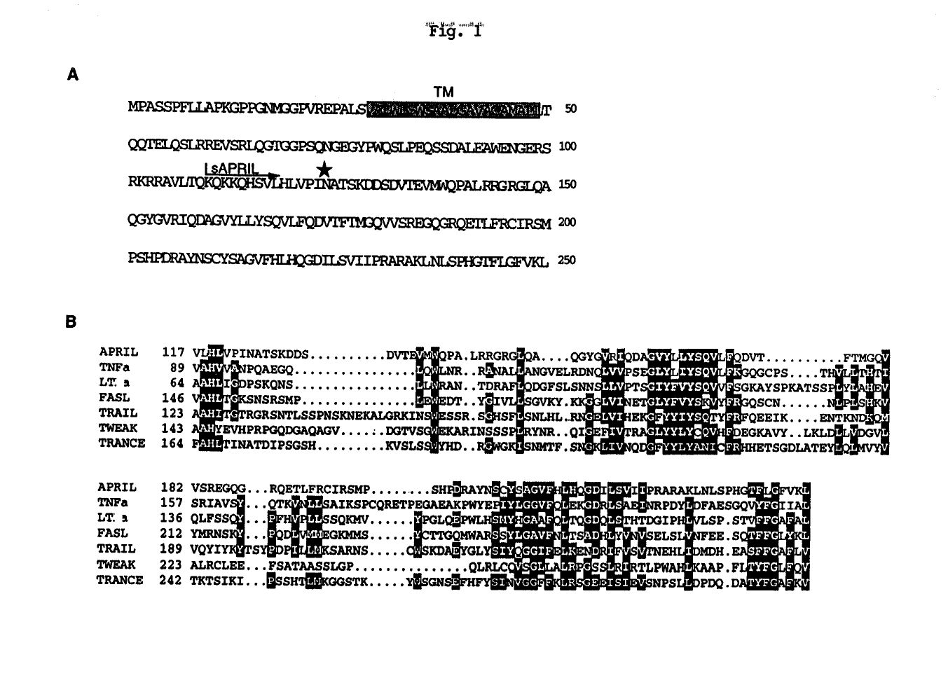

Comparison of the claimed APRIL sequence with other members of the human TNF family reveals considerable structural similarity. All the proteins share several regions of sequence conservation in the extracellular domain. The overall sequence homology of the extracellular domain of APRIL show the highest homology to FasL (21% amino acid identities), TNF α (20%), LT-α (18%), followed by TRAIL, TWEAK and TRANCE (15%). FIG. 1B.

The novel polypeptides of the invention specifically interact with a receptor, which has not yet been identified. However, the peptides and methods disclosed herein enable the identification of receptors which specifically interact with APRIL or fragments thereof.

The claimed invention in certain embodiments includes peptides derived from APRIL which have the ability to bind to its receptors. Fragments of APRIL can be produced in several ways, e.g., recombinantly, by PCR, proteolytic digestion or by chemical synthesis. Internal or terminal fragments of a polypeptide can be generated by removing one or more nucleotides from one end or both ends of a nucleic acid which encodes the polypeptide. Expression of the mutagenized DNA produces polypeptide fragments.

Polypeptide fragments can also be chemically synthesized using techniques known in the art such as conventional Merrifield solid phase f- moc or t-boc chemistry. For example, peptides and DNA sequences of the present invention may be arbitrarily divided into fragments of desired length with no overlap of the fragment, or divided into overlapping fragments of a desired length. Methods such as these are described in more detail below.

D. Generation of Soluble Forms of APRIL

Soluble forms of APRIL can often signal effectively and hence can be administered as a drug which now mimics the natural membrane form. It is possible that APRIL as claimed herein is naturally secreted as a soluble cytokines, however, if not, one can reengineer the gene to force secretion. To create a soluble secreted form of APRIL, one would remove at the DNA level the N-terminus transmembrane regions, and some portion of the stalk region, and replace them with a type I leader or alternatively a type II leader sequence that will allow efficient proteolytic cleavage in the chosen expression system. A skilled artisan could vary the amount of the stalk region retained in the secretion expression construct to optimize both receptor binding properties and secretion efficiency. For example, the constructs containing all possible stalk lengths, i.e. N-terminal truncations, could be prepared such that proteins starting at amino acids 18 to 139 would result. The optimal length stalk sequence would result from this type of analysis.

E. Generation of Antibodies Reactive with APRIL

The invention also includes antibodies specifically reactive with APRIL or its receptor. Anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal such as a mouse, a hamster or rabbit can be immunized with an immunogenic form of the peptide. Techniques for conferring immunogenicity on a protein or peptide include conjugation to carriers, or other techniques are well known in the art.

An immunogenic portion of APRIL or its receptor can be administered in the presence of an adjuvant. The progress of immunization can be monitored by detection of antibody titers in plasma or serum. Standard ELISA or other immunoassays can be used with the immunogen as antigen to assess the levels of antibodies.

In a preferred embodiment, the subject antibodies are immunospecific for antigenic determinants of APRIL, or its receptor, e.g. antigenic determinants of a polypeptide of SEQ. ID. NO.: 2, or a closely related human or non-human mammalian homolog (e.g. 70, 80 or 90 percent homologous, more preferably at least 95 percent homologous). In yet a further preferred embodiment of the present invention, the anti-APRIL or anti-APRIL-receptor antibodies do not substantially cross react (i.e. react specifically) with a protein which is e.g., less than 80 percent homologous to SEQ. ID. NO. 2; preferably less than 90 percent homologous with SEQ. ID. NO.: 2; and, most preferably less than 95 percent homologous with SEQ. ID. NO.: 2. By “not substantially cross react”, it is meant that the antibody has a binding affinity for a non-homologous protein which is less than 10 percent, more preferably less than 5 percent, and even more preferably less than 1 percent, of the binding affinity for a protein of SEQ. ID. NO. 2.

The term antibody as used herein is intended to include fragments thereof which are also specifically reactive with APRIL, or its receptor. Antibodies can be fragmented using conventional techniques and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments. The antibodies of the present invention are further intended to include biospecific and chimeric molecules having anti-APRIL or anti-APRIL -receptor activity. Thus, both monoclonal and polyclonal antibodies (Ab) directed against APRIL and its receptor, and antibody fragments such as Fab′ and F(ab′)₂, can be used to block the action of APRIL and its respective receptor.

Various forms of antibodies can also be made using standard recombinant DNA techniques. (Winter and Milstein, Nature 349: 293-299 (1991) specifically incorporated by reference herein.) For example, chimeric antibodies can be constructed in which the antigen binding domain from an animal antibody is linked to a human constant domain (e.g. Cabilly et al., U.S. Pat. No. 4,816,567, incorporated herein by reference). Chimeric antibodies may reduce the observed immunogenic responses elicited by animal antibodies when used in human clinical treatments.

In addition, recombinant “humanized antibodies” which recognize APRIL, or its receptor can be synthesized. Humanized antibodies are chimeras comprising mostly human IgG sequences into which the regions responsible for specific antigen-binding have been inserted. Animals are immunized with the desired antigen, the corresponding antibodies are isolated, and the portion of the variable region sequences responsible for specific antigen binding are removed. The animal-derived antigen binding regions are then cloned into the appropriate position of human antibody genes in which the antigen binding regions have been deleted. Humanized antibodies minimize the use of heterologous (i.e. inter species) sequences in human antibodies, and thus are less likely to elicit immune responses in the treated subject.

Construction of different classes of recombinant antibodies can also be accomplished by making chimeric or humanized antibodies comprising variable domains and human constant domains (CH1, CH2, CH3) isolated from different classes of immunoglobulins. For example, antibodies with increased antigen binding site valencies can be recombinantly produced by cloning the antigen binding site into vectors carrying the human chain constant regions. (Arulanandam et al., J. Exp. Med., 177: 1439-1450 (1993), incorporated herein by reference.)

In addition, standard recombinant DNA techniques can be used to alter the binding affinities of recombinant antibodies with their antigens by altering amino acid residues in the vicinity of the antigen binding sites. The antigen binding affinity of a humanized antibody can be increased by mutagenesis based on molecular modeling. (Queen et al., Proc. Natl. Acad. Sci. 86: 10029-33 (1989) incorporated herein by reference.

F. Generation of Analogs: Production of Altered DNA and Peptide Sequences

Analogs of APRIL can differ from the naturally occurring Ligands in amino acid sequence, or in ways that do not involve sequence, or both. Non-sequence modifications include in vivo or in vitro chemical derivatization of APRIL. Non-sequence modifications include, but are not limited to, changes in acetylation, methylation, phosphorylation, carboxylation or glycosylation.

Preferred analogs include, APRIL or biologically active fragments thereof, whose sequences differ from the sequence given in SEQ. ID NO. 2, by one or more conservative amino acid substitutions, or by one or more non-conservative amino acid substitutions, deletions or insertions which do not abolish the biological activity of APRIL. Conservative substitutions typically include the substitution of one amino acid for another with similar characteristics, e.g. substitutions within the following groups: valine, glycine; glycine, alanine; valine, isoleucine, leucine; aspartic acid, glutamic acid; asparagine, glutamine; serine, threonine; lysine, arginine; and, phenylalanine, tyrosine.

TABLE 1
CONSERVATIVE AMINO ACID REPLACEMENTS

| for amino Acid | code | replace with any of: |
|---|---|---|
| Alanine | A | D-Ala, Gly, Beta-Ala, L- |
| | | Cys, D-Cys |
| Arginine | R | D-Arg, Lys, D-Lys, homo- |
| | | Arg, D-homo-Arg, Met, |
| | | Ile, D-Met, D-Ile, Orn, D- |
| | | Orn |
| Asparagine | N | D-Asn, Asp, D-Asp, Glu, |
| | | D-Glu, Gln, D-Gln |
| Aspartic Acid | D | D-Asp, D-Asn, Asn, Glu, |
| | | D-Glu, Gln, D-Gln |
| Cysteine | C | D-Cys, S-Me-Cys, Met, D- |
| | | Met, Thr, D-Thr |
| Glutamine | Q | D-Gln, Asn, D-Asn, Glu, |
| | | D-Glu, Asp, D-Asp |
| Glutamic Acid | E | D-Glu, D-Asp, Asp, Asn, |
| | | D-Asn, Gln, D-Gln |
| Glycine | G | Ala, D-Ala, Pro, D-Pro, - |
| | | Ala, Acp |
| Isoleucine | I | D-Ile, Val, D-Val, Leu, D- |
| | | Leu, Met, D-Met |
| Leucine | L | D-Leu, Val, D-Val, Leu, |
| | | D-Leu, Met, D-Met |
| Lysine | K | D-Lys, Arg, D-Arg, |
| | | Homo-arg, D-homo-Arg, |
| | | Met, D-Met, Ile, D-Ile, |
| | | Orn, D-Orn |
| Methionine | M | D-Met, S-Me-Cys, Ile, D- |
| | | Ile, Leu, D-Leu, Val, D- |
| | | Val |
| Phenylalanine | F | D-Phe, Tyr, D-Thr, L- |
| | | Dopa, His, D-His, Trp, D- |
| | | Trp, Trans-3, 4 or 5- |
| | | phenylproline, cis-3, 4, or |
| | | 5-phenylproline |
| Proline | P | D-Pro, L-I-thoazolidine-4- |
| | | carboxylic acid, D- or L-1- |
| | | oxazolidine-4-carboxylic |
| | | acid |
| Serine | S | D-Ser, Thr, D-Thr, allo- |
| | | Thr, Met, D-Met, Met(O), |
| | | D-Met(O), L-Cys, D-Cys |
| Threonine | T | D-Thr, Ser, D-Ser, allo- |
| | | Thr, Met, D-Met, Met(O), |
| | | D-Met(O), Val, D-Val |
| Tyrosine | Y | D-Tyr, Phe, D-Phe, L- |
| | | Dopa, His, D-His |
| Valine | V | D-Val, Leu, D-Leu, Ile, D- |
| | | Ile, Met, D-Met |

Useful methods for mutagenesis include PCR mutagenesis and saturation mutagenesis as discussed in more detail below. A library of random amino acid sequence variants can also be generated by the synthesis of a set of degenerate oligonucleotide sequences.

PCR Mutagenesis

In PCR mutagenesis, reduced Taq polymerase fidelity can be used to introduce random mutations into a cloned fragment of DNA (Leung et al., 1989, Technique 1:11-15). This is a very powerful and relatively rapid method of introducing random mutations. The DNA region to be mutagenized can be amplified using the polymerase chain reaction (PCR) under conditions that reduce the fidelity of DNA synthesis by Taq DNA polymerase, e.g., by using a dGTP/dATP ratio of five and adding Mn²⁺ to the PCR reaction. The pool of amplified DNA fragments can be inserted into appropriate cloning vectors to provide random mutant libraries.

Saturation Mutagenesis

Saturation mutagenesis allows for the rapid introduction of a large number of single base substitutions into cloned DNA fragments (Mayers et al., 1985, Science 229:242). This technique includes generation of mutations, e.g., by chemical treatment or irradiation of single-stranded DNA in vitro, and synthesis of a complimentary DNA strand. The mutation frequency can be modulated by modulating the severity of the treatment, and essentially all possible base substitutions can be obtained. Because this procedure does not involve a genetic selection for mutant fragments both neutral substitutions, as well as of a protein can be prepared by random mutagenesis of DNA which those that alter function, can be obtained. The distribution of point mutations is not biased toward conserved sequence elements.

Degenerate Oligonucleotides

A library of homologs can also be generated from a set of degenerate oligonucleotide sequences. Chemical synthesis of degenerate sequences can be carried out in an automatic DNA synthesizer, and the synthetic genes then ligated into an appropriate expression vector. The synthesis of degenerate oligonucleotides is known in the art (See for example, Narang, SA (1983) Tetrahedron 39:3; Itakura et al. (1981) Recombinant DNA, Proc 3rd Cleveland Sympos. Macromolecules, ed. AG Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (See, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Non-random or directed, mutagenesis techniques can be used to provide specific sequences or mutations in specific regions. These techniques can be used to create variants which include, e.g., deletions, insertions, or substitutions, of residues of the known amino acid sequence of a protein. The sites for mutation can be modified individually or in series, e.g., by (1) substituting first with conserved amino acids and then with more radical choices depending upon results achieved, (2) deleting the target residue, or (3) inserting residues of the same or a different class adjacent to the located site, or combinations of options 1-3.

Alanine Scanning Mutagenesis

Alanine scanning mutagenesis is a useful method for identification of certain residues or regions of the desired protein that are preferred locations or domains for mutagenesis, Cunningham and Wells (Science 244:1081-1085, 1989) specifically incorporated by reference. In alanine scanning, a residue or group of target residues are identified (e.g., charged residues such as Arg, Asp, His, Lys, and Glu) and replaced by a neutral or negatively charged amino acid (most preferably alanine or polyalanine). Replacement of an amino acid can affect the interaction of the amino acids with the surrounding aqueous environment in or outside the cell. Those domains demonstrating functional sensitivity to the substitutions can then be refined by introducing further or other variants at or for the sites of substitution. Thus, while the site for introducing an amino acid sequence variation is predetermined, the nature of the mutation per se need not be predetermined. For example, to optimize the performance of a mutation at a given site, alanine scanning or random mutagenesis may be conducted at the target codon or region and the expressed desired protein subunit variants are screened for the optimal combination of desired activity.

Oligonucleotide-Mediated Mutagenesis

Oligonucleotide-mediated mutagenesis is a useful method for preparing substitution, deletion, and insertion variants of DNA, see, e.g., Adelman et al., (DNA 2:183, 1983) incorporated herein by reference. Briefly, the desired DNA can be altered by hybridizing an oligonucleotide encoding a mutation to a DNA template, where the template is the single-stranded form of a plasmid or bacteriophage containing the unaltered or native DNA sequence of the desired protein. After hybridization, a DNA polymerase is used to synthesize an entire second complementary strand of the template that will thus incorporate the oligonucleotide primer, and will code for the selected alteration in the desired protein DNA. Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al. (Proc. Natl. Acad. Sci. USA, 75: 5765[1978]) incorporated herein by reference.

Cassette Mutagenesis

Another method for preparing variants, cassette mutagenesis, is based on the technique described by Wells et al. (Gene, 34:315[1985]) incorporated herein by reference. The starting material can be a plasmid (or other vector) which includes the protein subunit DNA to be mutated. The codon(s) in the protein subunit DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the desired protein subunit DNA. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are comparable with the ends of the linearized plasmid, such that it can be directly ligated to the plasmid. This plasmid now contains the mutated desired protein subunit DNA sequence.

Combinatorial Mutagenesis

Combinatorial mutagenesis can also be used to generate mutants. E.g., the amino acid sequences for a group of homologs or other related proteins are aligned, preferably to promote the highest homology possible. All of the amino acids which appear at a given position of the aligned sequences can be selected to create a degenerate set of combinatorial sequences. The variegated library of variants is generated by combinatorial mutagenesis at the nucleic acid level, and is encoded by a variegated gene library. For example, a mixture of synthetic oligonucleotides can be enzymatically ligated into gene sequences such that the degenerate set of potential sequences are expressible as individual peptides, or alternatively, as a set of larger fusion proteins containing the set of degenerate sequences.

Various techniques are known in the art for screening generated mutant gene products. Techniques for screening large gene libraries often include cloning the gene library into replicable expression vectors, transforming appropriate cells with the resulting library of vectors, and expressing the genes under conditions in which detection of a desired activity, e.g., in this case, binding to APRIL or its receptor, facilitates relatively easy isolation of the vector encoding the gene whose product was detected. Each of the techniques described below is amenable to high through-put analysis for screening large numbers of sequences created, e.g., by random mutagenesis techniques.

The invention also provides for reduction of the protein binding domains of the claimed polypeptides or their receptors, to generate mimetics, e.g. peptide or non-peptide agents. The peptide mimetics are able to disrupt binding of APRIL with its respective receptor. The critical residues of the APRIL involved in molecular recognition of a receptor polypeptide or of a downstream intracellular protein, can be determined and used to generate APRIL or its receptor-derived peptidomimetics which competitively or noncompetitively inhibit binding of APRIL with a receptor. (see, for example, “Peptide inhibitors of human papilloma virus protein binding to retinoblastoma gene protein” European patent applications EP-412,762A and EP-B31,080A), specifically incorporated herein by reference.

By making available purified and recombinant APRIL, the present invention provides assays which can be used to screen for drug candidates which are either agonists or antagonists of the normal cellular function, in this case, of APRIL or its receptor. In one embodiment, the assay evaluates the ability of a compound to modulate binding between APRIL and a receptor. A variety of assay formats will suffice and, in light of the present inventions, will be comprehended by the skilled artisan.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or change in enzymatic properties of the molecular target. Isolation of a receptor binding to APRIL

Ligands of the TNF family can be used to identify and clone receptors. With the described APRIL sequences, one could fuse the 5′ end of the extracellular domain which constitutes the receptor binding sequence to a marker or tagging sequence and then add a leader sequence that will force secretion of APRIL in any of a number of expression systems. One example of this technology is described by Browning et al., (1996) (JBC 271, 8618-8626) where the LT-β ligand was secreted in such a form. The VCAM leader sequence was coupled to a short myc peptide tag followed by the extracellular domain of the LT-β. The VCAM sequence is used to force secretion of the normally membrane bound LT-β molecule. The secreted protein retains a myc tag on the N-terminus which does not impair the ability to bind to a receptor. Such a secreted protein can be expressed in either transiently transfected Cos cells or a similar system, e.g., EBNA derived vectors, insect cell/baculovirus, picchia etc. The unpurified cell supernatant can be used as a source of the tagged ligand.

Cells expressing the receptor can be identified by exposing them to the tagged ligand. Cells with bound ligand are identified in a FACS experiment by labeling the myc tag with an anti-myc peptide antibody (9E10) followed by phycoerythrin (or a similar label)labeled anti-mouse immunoglobulin. FACS positive cells can be readily identified and would serve as a source of RNA encoding for the receptor. An expression library would then be prepared from this RNA via standard techniques and separated into pools: Pools of clones would be transfected into a suitable host cell and binding of the tagged ligand to receptor positive transfected cells determined via microscopic examination, following labeling of bound myc peptide tag with an enzyme labeled anti-mouse Ig reagent, i.e. galactosidase, alkaline phosphatase or luciferase labeled antibody. Once a positive pool has been identified, the pool size would be reduced until the receptor encoding cDNA is identified. This procedure could be carried out with either the mouse or human APRIL, as one may more readily lead to a receptor.

G. METHODS OF TREATMENT AND PHARMACEUTICAL COMPOSITIONS

The methods of the invention for the treatment of cancers involve the administration to a patient, preferably a mammalian host, such as a dog, cat, or human, an effective amount of a claimed composition comprising a blocking agent capable of interfering with the association between APRIL and its receptor. Such blocking agents include, but are not limited to soluble APRIL, anti-APRIL antibodies, anti-APRIL receptor antibodies, or biologically active fragments thereof. Additionally, an inhibitory form of APRIL can be made by mutating APRIL, while maintaining the ability to block the association between APRIL and its receptor. Blocking agents may preferably comprise a receptor IG fusion protein, which can be constructed by methods known to those of skill in the art.

The methods of the invention are useful for treating all cancers, including, but not limited to, cellular disorders as, for example, renal cell cancer, Kaposi's sarcoma, chronic leukemia, breast cancer, sarcoma, ovarian carcinoma, rectal cancer, throat cancer, melanoma, colon cancer, bladder cancer, mastocytoma, lung cancerm mammary adenocarcinoma, pharyngeal squamous cell carcinoma, and gastrointestinal or stomach cancer. Additionally, such blocking agents are useful for the treatment of proliferative conditions that are not considered to be tumors, i.e. cellular hyperproliferation (hyperplasia), such as, for example, scleroderma, pannus formation in rheumatoid arthritis, postsurgical scarring and lung, liver and uterine fibrosis.

Pharmaceutical compositions of the invention may comprise a therapeutically effective amount of APRIL, or its receptor, or fragments or mimetics thereof, and, optionally may include pharmaceutically acceptable carriers. Accordingly, this invention provides methods for treatment of cancer, and methods of stimulating, or in certain instances, inhibiting the immune system, or parts thereof by administering a pharmaceutically effective amount of a compound of the invention or its pharmaceutically acceptable salts or derivatives. It should of course by understood that the compositions and methods of this invention can be used in combination with other therapies for various treatments.

The compositions can be formulated for a variety of routes of administration, including systemic, topical or localized administration. For systemic administration, injection is preferred, including intramuscular, intravenous, intraperitoneal, and subcutaneous for injection, the compositions of the invention can be formulated in liquid solutions, preferably in physiologically compatible buffers such as Hank's solution or Ringer's solution. In addition, the compositions may be formulated in solid form and, optionally, redissolved or suspended immediately prior to use. Lyophilized forms are also included in the invention.

The compositions can be administered orally, or by transmucosal or transdermal means. For transmucosal or transdermal administration, penetrants appropriate to the barrier to be permeated are used in the formulation. Such penetrants are known in the art, and include, for example, for transmucosal administration, bile salts, fusidic acid derivatives, and detergents. Transmucosal administration may be through nasal sprays or using suppositories. For oral administration, the compositions are formulated into conventional oral administration forms such as capsules, tablets, and tonics. For topical administration, the compositions of the invention are formulated into ointments, salves, gels, or creams as known in the art.

The dose and dosing regimen will depend on the type of cancer, the patient and the patient's history. The amount must be effective to treat, suppress, or alter the progression of cancer. The doses may be single doses or multiple doses. If multiple doses are employed, as preferred, the freuence of administration will depend, for rexample, on the type of host and and type of cancer, dosage amounts etc. For some types of cancers or cancer lines, dily administration will be effective, whereas for others, administration every other day or every third day will be effective. The amount of active compound administered at one time or over the course of treatment will depend on many factors. For example, the age and size of the subject, the severity and course of the disease being treated, the manner and form of administration, and the judgments of the treating physician However, an -effective dose may be in the range of from about 0.005 to about 5 mg/kg/day, preferably about 0.05 to about 0.5 mg/kg/day. The dosage amount which will be most effective will be one which results in no tumor appearance or complete regression of the tumor, and is not toxic to the patient. One skilled in the art will recognize that lower and higher doses may also be useful.

Gene constructs according to the invention can also be used as a part of a gene therapy protocol to deliver nucleic acids encoding either an agonistic or antagonistic form of APRIL.

Expression constructs of the APRIL can be administered in any biologically effective carrier, e.g., any formulation or composition capable of effectively delivering the gene for APRIL to cells in vivo. Approaches include insertion of the gene in viral vectors which can transfect cells directly, or delivering plasmid DNA with the help of, for example, liposomes, or intracellular carriers, as well as direct injection of the gene construct. Viral vector transfer methods are preferred.

A pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced intact from recombinant cells, e.g. retroviral vectors, the pharmaceutical preparation can comprise one or more cells which produce the gene delivery system.

In addition to use in therapy, the oligomers of the invention may be used as diagnostic reagents to detect the presence or absence of the target DNA, RNA or amino acid sequences to which they specifically bind. In other aspects, the claimed invention may be used to evaluate a chemical entity for its ability to interact with, e.g., bind or physically associate with APRIL or a fragment thereof. The method includes contacting the chemical entity with APRIL, and evaluating the ability of the entity to interact with APRIL. Additionally, APRIL can be used in methods of evaluating naturally occurring APRIL or receptors of APRIL, as well as to evaluate chemical entities which associate or bind with receptors of APRIL. It may be desirable to use tagged versions of APRIL to facilitate the detection of APRIL binding to its receptor, or receptor positive cells, such as, for example, the purpose of screening for agents that block the APRIL ligand-APRIL receptor interaction. Additionally, one may use APRIL transfected cell lines that have increased growth rates as the basis for screening assays for molecules that block APRIL activity.

In certain aspects, the claimed invention features a method for evaluating a chemical entity for the ability to modulate the interaction between APRIL and its respective receptor. The method includes combining a receptor for APRIL, and APRIL under conditions wherein the pair is capable of interacting, adding the chemical entity to be evaluated and detecting the formation or dissolution of complexes. These modulating agents may be further evaluated in vitro, e.g. by testing its activity in a cell free system, and then, optionally administering the compound to a cell or animal, and evaluating the effect.

### I. EXAMPLES

### Example 1

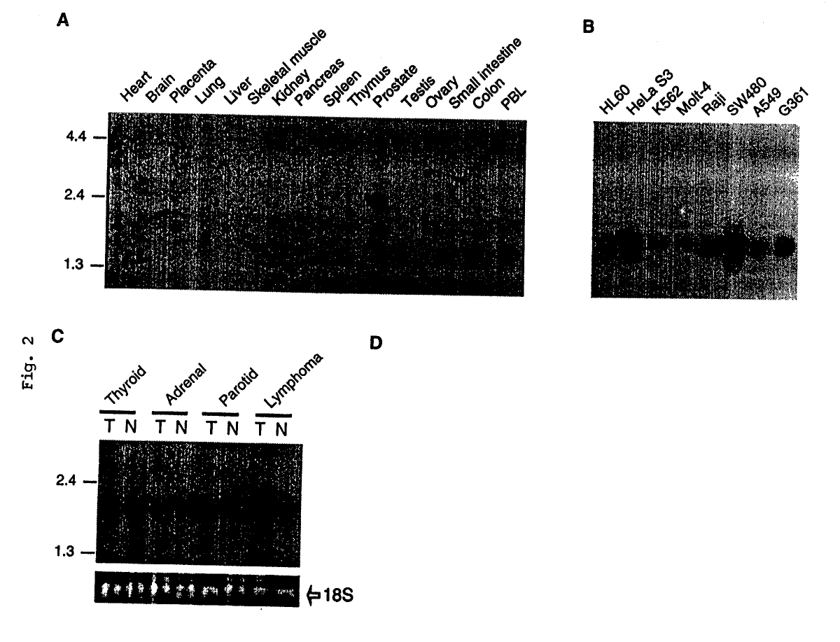

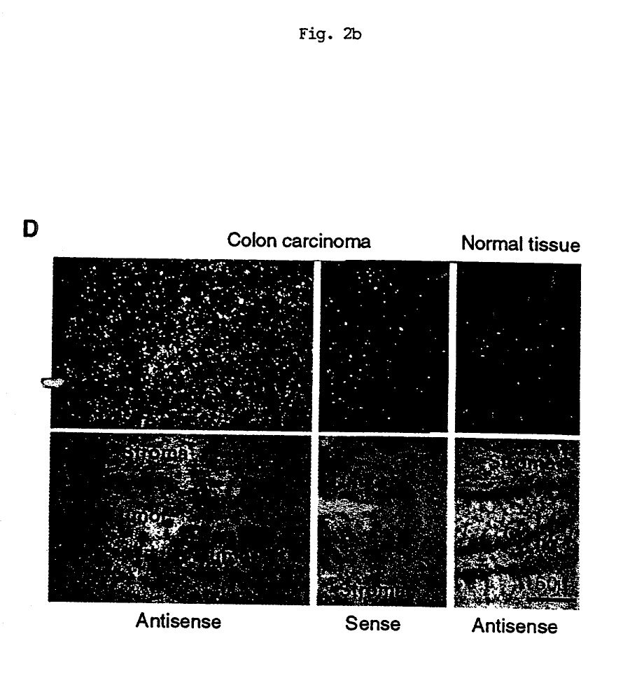

Northern blot analysis of APRIL revealed that the expression of APRIL was weak and restricted only to a few tissues (FIG. 2A). Two transcripts of 2.1 kb and 2.4 kb were found in the prostate, whereas PBLs revealed a shorter transcript of 1.8 kb. Northern blot analysis was performed by using Human Multiple Tissue Northern Blots I and II (Clontech #7760-1 and #7759-1), Human Cancer Cell Line MTN Blot (Clontech #7757-1) and Human Tumor Panel Blot V (Invitrogen D3500-01). The membranes were incubated in ExpressHyb hybridization solution (Clontech #8015-1) for at least 1 hour at 62° C. The random-primed cDNA probe (Boehringer Mannheim) was synthesized using cDNA corresponding to the extracellular domain of APRIL as template. The heat-denatured cDNA probe was added at 1.5×10⁶ cpm/ml in fresh ExpressHyb. The membrane was hybridized 12-24 hr at 62° C., washed three times in 2×SSC containing 0.05% SDS and exposed at −70° C. Northern blot analysis of APRIL revealed that the expression of APRIL was weak and restricted only to a few tissues. Two transcripts of 2.1 kb and 2.4 kb were found in th eprostrate, whereas PBLs revealed a shorter transcript of 1.8 kb.

A longer exposure time revealed the 2.1 kb APRIL mRNA in colon, spleen, and pancreas (data not shown). This restricted distribution of the APRIL mRNA is consistent with the origin of cDNA clones currently available in the EST database. Of the 23 clones identified only two were derived from normal tissues (pregnant uterus, pancreatic islands). Remarkably, the remainder of the EST-clones (21 clones, 91%) were present in cDNA libraries generated from tumors or tumor-derived cell lines (Ovary tumor, 11; prostate tumor, 3; Gessler Wilms tumor, 1; colon carcinoma, 1; endometrial tumor, 1; parathyroid tumors, 1; pancreas tumor, 1; T-cell lymphoma, 1; LNCAP adenocarcinoma derived cell line, 1). This prompted us to test transformed cell lines for the expression of APRIL mRNA (FIG. 2B), and indeed, all cell lines strongly expressed the 2.1 kb transcript of APRIL.

Highest APRIL-specific signals were detected in the colorectal adenocarcinoma SW480, the Burkiu's lymphoma Raji and in the melanoma G361. To corroborate this finding, we measured APRIL mRNA expression levels in several tumors and compared them to normal tissues. APRIL mRNA was abundantly detected in thyroid carcinoma and in lymphoma, whereas in the corresponding normal tissues, only weak or no hybridization signals were found (FIG. 2C). In the two other tumors nalyzed by Northern blots (adrenal and paratoid tumors), APRIL mRNA was not elevated. However, in situ hybridization revealed abundant APRIL message in human colon adenocarcinoma as compared to normal colon tissue (FIG. 2D).

In order to explore possible activities of APRIL, we expressed a recombinant form of soluble extracellular domain of APRIL (sAPRIL) encompassing amino acids 110 to 250 in 293 cells (9). The full length APRIL gene was amplified from the EST-clone, using a specific 5 ′ forward primer flanked by a EcoRI site (5′-CCAGCCTCATCTCCTTTCTTGC-3′) and a specific 3′ reverse primer flanked by an XbaI site (5′-TCACAGTTTCACAAACCCCAGG-3′). The amplified fragment was cut with EcoRI/LXbaI and cloned into a modified version of pCRIII (Invitrogen), in frame with an N-terminal Flag peptide (15). The soluble form of APRIL (sAPRIL) was generated using the two primers (5′-AAACAGAAGAAGCAGCACTCTG-3′) and (5′-TCACAGTTTCACAAACCCCAGG-3′) containing a PstI and XbaI site, respectively, and subsequently cloned into a modified pCRIII vector, containing both a HA signal for protein secretion in eukaryotic cells and an N-terminal Flag epitope (15).

### Example 2

The widespread expression of APRIL in tumor cells and tissues suggested to us that APRIL may be associated with tumor growth, and we therefore incubated various tumor cell lines with purified recombinant Flag-tagged sAPRIL (10).

Human embryonic 293T cells, human leukemia Jurkat T-cells, human Burkitt lymphoma B-cells Raji and melanoma cell lines were grown as previously described (16, 17). Other cell lines referred in this paper are deposited in and described by the American Type Culture Collection (Rockville, Maryland). All cell lines were cultured in RPMI or DMEM medium supplemented with 10% fetal calf serum.

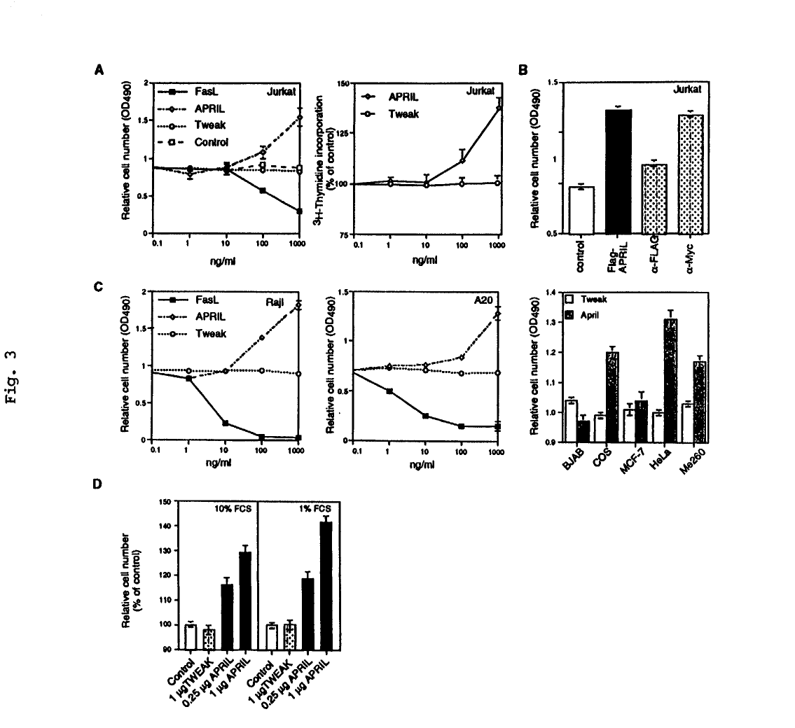

Flag-tagged versions of the extracellular domain (residues 103-28 1) of human FasL and TRAIL (residues 95-281) were recently described (15). Flag-tagged soluble human TWEAK (residues 141-284) was produced in 293 cells (P. S. manuscript in preparation). The anti-Flag antibody M2 were obtained from Kodak International Biotechnologies. An increase in proliferation of the Jurkat T lymphoma cells in the presence of APRIL was observed in a dose dependent manner as detected by an increase in number (approximately 50%) (11) of viable cells 24 hrs after ligand addition (FIG. 3A). The proliferation of cells was determined by incubating cells at 50,000 cells per well in 100 μl medium with the indicated concentrations of recombinant APRIL, TWEAK, TRAIL, FasL and by determining the number of viable cells using the Celltiter 96 AQ proliferation assay (Promega) after 24 hrs, following the manufacturer's instructions, or by ³H-thymidine incorporation. For the immunodepletion of Flag-APRIL anti-Flag coupled to agarose was used.

The increase in proliferation was independent of a co-stimulatory signals such as anti-CD3 antibodies or other cytokines. As expected, the addition of identically produced and purified FasL to Jurkat cells decreased the number of viable cells, whereas TWEAK had no effect. The increased cell number correlated with augmented (40%) ³H-thymidine incorporation in APRIL-treated cells (FIG. 3A). Immunodepletion of FLAG-tagged APRIL -containing conditioned medium by anti-FLAG antibodies, but not anti-myc antibodies, reduced the proliferative effect (FIG. 3B), indicating that the proliferative effect was specific and due to APRIL. Increased proliferation rates were also seen in some B lymphomas (human Raji, mouse A20 cells, but not human BJAB) and on cell lines of epithelial origin such as COS and HeLa, as well as melanomas (FIG. 3C). The breast carcinoma cell MCF-7 did not respond. The effect on Jurkat cells was even more pronounced when the fetal calf serum was reduced from 10% to 1% (FIG. 3D).

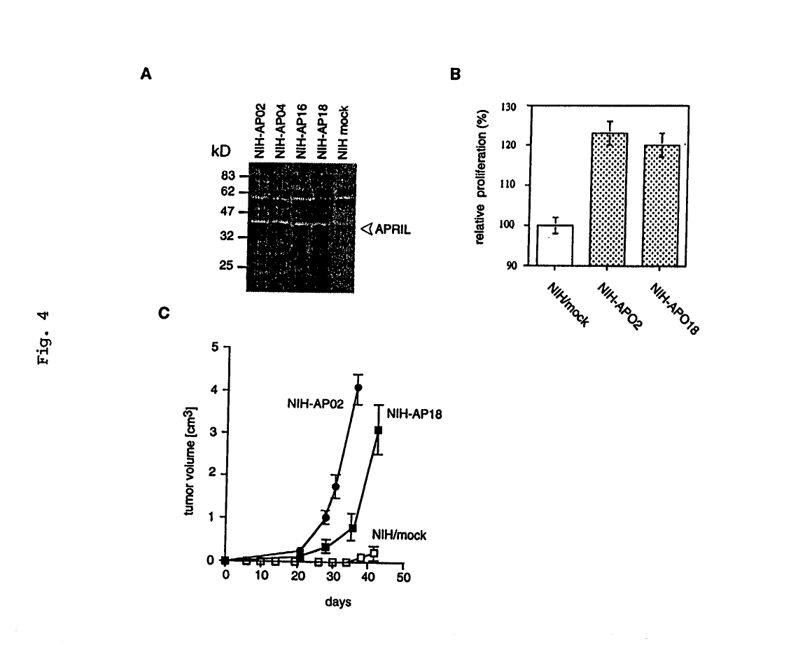

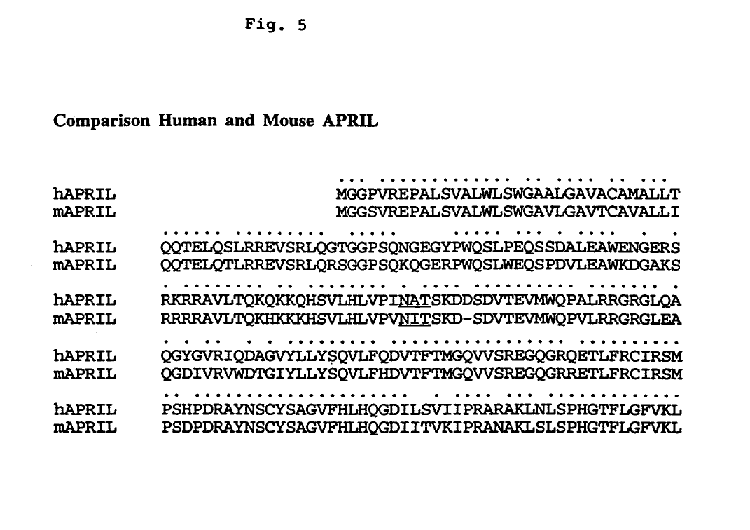

Recombinant sAPRIL has forms aggregates which may explain the rather high concentrations needed to detect a proliferative effect with sAPRIL. We therefore transfected NIH-3T3 cells with full-length human APRIL (12) and obtained several APRIL-expressing clones (FIG. 4A). NIH-3T3 APRIL clones were established using the calcium phosphate method of transfection and the full-length FLAG-tagged APRIL containing pCRIII expression vector. Cellular proteins of about 2×10⁶ cells per lane were electrophoretically separated on a 12% polyacrylamide gel in the presence of SDS under reducing conditions and subsequently transferred to nitrocellulose. Immunoblot analysis of Flag-tagged APRIL was conducted using 5 μg/ml of the rat monoclonal anti-Flag antibody M2 (Kodak International Biotechnologies). First antibodies were detected using affinity purified anti-peroxidase-conjugated donkey anti-mouse antibody (Dianova, Hamburg, Germany) followed by a chemiluminescence reaction using the ECL system (Amersham).

Interestingly, APRIL transfectants proliferated faster than mock-transfectants (FIG. 4B). We reasoned that the APRIL-transfected NIH-3T3 cells might also have a growth advantage in vivo. When wild-type or mock-transfected NIH-3T3 cells were injected into nude mice, small palpable tumors were observed after 5-6 weeks (13). In contrast, two clones of NIH-3T3 cells stably transfected with APRIL both induced tumors after only 3-4 weeks. After 6 weeks, mice had to be killed due to the high tumor burden (FIG. 4C). NIH V3T3 fibroblasts (American Type Culture Collection, Rockville, Maryland) and the various transfectants (1×10⁵ cells) were suspended in 50 μl PBS and injected subcutaneously into the flank region of BALB/c nude mice (Harlan, Zeist, Netherland). Tumor size was measured every three days. Mice were age-matched (3 animals per group).

### Example 3

Isolation of a receptor binding to APRIL.

Ligands of the TNF family can be used to identify and clone receptors. With the described APRIL sequences, one could fuse the 5′ end of the extracellular domain of APRIL which constitutes the receptor binding sequence to a marker or tagging sequence and then add a leader sequence that will force secretion of APRIL in any of a number of expression systems. One example of this technology is described by Browning et al., (1996) (JBC 271, 8618-8626) where the LT-β ligand was secreted in such a form. The VCAM leader sequence was coupled to a short myc peptide tag followed by the extracellular domain of the LT-β. The VCAM sequence is used to force secretion of the normally membrane bound LT-β molecule. The secreted protein retains a myc tag on the N-terminus which does not impair the ability to bind to a receptor. Such a secreted protein can be expressed in either transiently transfected Cos cells or a similar system, e.g., EBNA derived vectors, insect cell/baculovirus, picchia etc. The unpurified cell supernatant can be used as a source of the tagged ligand.

Cells expressing the receptor can be identified by exposing them to the tagged ligand. Cells with bound ligand are identified in a FACS experiment by labeling the myc tag with an anti-myc peptide antibody (9E10) followed by phycoerythrin (or a similar label) labeled anti-mouse immunoglobulin. FACS positive cells can be readily identified and would serve as a source of RNA encoding for the receptor. An expression library would then be prepared from this RNA via standard techniques and separated into pools. Pools of clones would be transfected into a suitable host cell and binding of the tagged ligand to receptor positive transfected cells determined via microscopic examination, following labeling of bound myc peptide tag with an enzyme labeled anti-mouse Ig reagent, i.e. galactosidase, alkaline phosphatase or luciferase labeled antibody. Once a positive pool has been identified, the pool size would be reduced until the receptor encoding cDNA is identified. This procedure could be carried out with either the mouse or human APRIL, as one may more readily lead to a receptor.

It will be apparent to those skilled in the art that various modifications and variations can be made in APRIL, compositions and methods of the present invention without departing from the spirit or scope of the invention. Thus, it is intended that the present invention cover the modifications and variations of this invention provided that they come within the scope of the appended claims and their equivalents.

| | | |
|---|---|---|
| 1 | GGTACGAGGC TCCTAGAGG GACTGGAACCTAATTCTCCT GAGGCTGAGG | SEQ ID NO:1 |
| 51 | GAGGGTGGAG GGTCTCAAGG CAACGCTGGC CCCACGACGG AGTGCCAGGA | |
| 101 | GCACTAACAG TACCCTTAGC TTGCTTTCCT CCTCCCTCCT TTTTATTTTC | |
| 151 | AAGTTCCTTT TTATTTCTCC TTGCGTAACA ACCTTCTTCC CTTCTGCACC | |
| 201 | ACTGCCCGTA CCCTTACCCG CCCCGCCACC TCCTTGCTAC CCCACTCTTG | |
| 251 | AAACCACAGC TGTTGGCAGG GTCCCCAGCT CATGCCAGCC TCATCTCCTT | |
| 301 | TCTTGCTAGC CCCCAAAGGG CCTCCAGGCA ACATGGGGGG CCCAGTCAGA | |
| 351 | GAGCCGGCAC TCTCAGTTGC CCTCTGGT G AGTTGGGGGG CAGCTCTGGG | |
| 401 | GGCCGTGGCT TGTGCCATGG CTCTGCTGAC CCAACAAACA GAGCTGCAGA | |
| 451 | GCCTCAGGAG AGAGGTGAGC CGGCTGCAGG GGACAGGAGG CCCTCCCAG | |
| 501 | AATGGGGAAG GGTATCCCTG GCAGAGTCTC CCGGAGCAGA GTTCCGATGC | |
| 551 | CCTGGAAGCC TGGGAGAATG GGGAGAGATC CCGGAAAAGG GAGCAGTGC | |
| 601 | TCACCCAAAA ACAGAAGAAG CAGCACTCTG TCCTGCACCT GGTTCCCATT | |
| 651 | AACGCCACCT CCAAGGATGA CTCCGATGTG ACAGAGGTGA TGTGGCAACC | |
| 701 | AGCTCTTAGG CGTGGGAGAG GCCTACAGGC CCAAGGATAT GGTGTCCGAA | |
| 751 | TCCAGGATGC TGGAGTTTAT CTGCTGTATA GCCAGGTCCT GTTTCAAGAC | |
| 801 | GTGACTTTCA CCATGGGTCA GGTGGTGTCT CGAGAAGGCC AAGGAAGGCA | |
| 851 | GGAGACTCTA TTCCGATGTA TAAGAAGTAT GCCCTCCCAC CCGGACCGGG | |
| 901 | CCTACAACAG CTGCTATAGC GCAGGTGTCT TCCATTTACA CCAAGGGGAT | |
| 951 | ATTCTGAGTG TCATAATTCC CCGGGCAAGG GCGAAACTTA ACCTCTCTCC | |
| 1001 | ACATGGAACC TTCCTGGGGT TTGTGAAACT GTGATTGTGT TATAAAAAGT | |
| 1051 | GGCTCCCAGC TTGGAAGACC AGGGTGGGTA CATACTGGAGACAGCCAAGA | |
| 1101 | GCTGAGTATA TAAAGGAGAG GGAATGTGCA GGAACAGAGGCATCTTCCTG | |
| 1151 | GGTTTGGCTC CCCGTTCCTC ACTTTTCCCT TTTCATTCCC ACCCCCTAGA | |
| 1201 | CTTTGATTTT ACGGATATCT TGCTTCTGTT CCCCATGGAG CTCCGAATTC | |
| 1251 | TTGCGTGTGT GTAGATGAGG GGCGGGGGAC GGGCGCCAGG CATTGTTCAG | |
| 1301 | ACCTGGTCGG GGCCCACTGG AAGCATCCAG AACAGCACCA CCATCTTA | |
| 1 | MPASSPFLLA PKGPPGNMGG PVREPALSVA LWLSWGAALG AVACAMALLT | SEQ ID NO:2 |
| 51 | QQTELQSLRR EVSRLQGTGG PSQNGEGYPW QSLPEQSSDA LEAWENGERS | |
| 101 | RKRRAVLTQK QKKQHSVLHL VPINATSKDD SDVTEVMWQP ALRRGRGLQA | |
| 151 | QGYGVRIQDA GVYLLYSQVL FQDVTFTMGQ VVSREGQGRQ ETLFRCIRSM | |
| 201 | PSHPDRAYNS CYSAGVFHLH QGDILSVIIP RARAKLNLSP HGTFLGFVKL | |
| 1 | GAATTCGGCA CGAGGCTCCA GGCCACATGG GGGGCTCAGT CAGAGAGCCA | SEQ ID NO: 3 |
| 51 | GCCCTTTCGG TTGCTCTTTG GTTGAGTTGG GGGGCAGTTC TGGGGGCTGT | |
| 101 | GACTTGTGCT GTCGCACTAC TGATCCAACA GACAGAGCTG CAAAGCCTAA | |
| 151 | GGCGGGAGGT GAGCCGGCTG CAGCGGAGTG GAGGGCCTTC CCAGAAGCAG | |
| 201 | GGAGAGCGCC CATGGCAGAG CCTCTGGGAG CAGAGTCCTG ATGTCCTGGA | |
| 251 | AGCCTGGAAG GATGGGGCGA AATCTCGGAG AAGGAGAGCA GTACTCACCC | |
| 301 | AGAAGCACAA GAAGAAGCAC TCAGTCCTGC ATCTTGTTCC AGTTAACATT | |
| 351 | ACCTCCAAGG ACTCTGACGT GACAGAGGTG ATGTGGCAAC CAGTACTTAG | |
| 401 | GCGTGGGAGA GGCCCTGGAG GCCCAGGGAG ACATTGTACG AGTCTGGGAC | |
| 451 | ACTGGAATTT ATCTGCTCTA TAGTCAGGTC CTGTTTCATG ATGTGACTTT | |
| 501 | CACAATGGGT CAGGTGGTAT CTCGGGAAGG ACAAGGGAGA AGAGAAACTC | |
| 551 | TATTCCGATG TATCAGAAGT ATGCCTTCTG ATCCTGACCG TGCCTACAAT | |
| 601 | AGCTGCTACA GTGCAGGTGT CTTTCATTTA CATCAAGGGG ATATTATCAC | |
| 651 | TGTCAAAATT CCACGGGCAA ACGCAAAACT TAGCCTTTCT CCGCATGGAA | |
| 701 | CATTCCTGGG GTTTGTGAAA CTATGATTGT TATAAAGGGG GTGGGGATTT | |
| 751 | CCCATTCCAA AAACTGGCTA GACAAAGGAC AAGGAACGGT CAAGAACAGC | |
| 801 | TCTCCATGGC TTTGCCTTGA CTGTTGTTCC TCCCTTTGCC TTTCCCGCTC | |
| 851 | CCACTATCTG GGCTTTGACT CCATGGATAT TAAAAAAGTA GAATATTTTG | |
| 901 | TGTTTATCTC CCAAAAA | |
| 1 | MGGSVREPAL SVALWLSWGA VLGAVTCAVA LLIQQTELQS LRREVSRLQR | SEQ ID NO:4 |
| 51 | SGGPSQKQGE RPWQSLWEQS PDVLEAWKDG AKSRRRRAVL TQKHKKKHSV | |
| 101 | LHLVPVNITS KDSDVTEVMW QPVLRRGRGP GGQGDIVRVW DTGIYLLYSQ | |
| 151 | VLFHDVTFTM GQVVSREGQG RRETLFRCIR SMPSDPDRAY NSCYSAGVFH | |
| 201 | LHQGDIITVK IPRANAKLSL SPHGTFLGFV KL | |

What is claimed is:

1. Nucleic acid encoding an APRIL ligand, said ligand comprising a polypeptide of at least about 102 amino acids.

2. Nucleic acid of claim 1 encoding SEQ ID No: 2.

3. Nucleic acid encoding an APRIL ligand or a fragment thereof, wherein said ligand comprises an amino acid sequence found in amino acids 1 to 55 of SEQ ID NO: 2.

4. Nucleic acid encoding an APRIL ligand or a fragment thereof, wherein said ligand comprises an amino acid sequence found in amino acids 157 to 250 of SEQ ID NO: 2.

5. Nucleic acid of claims 1-4 encoding an amino acid substitution analog of SEQ ID No: 2.

6. Nucleic acid of claim 5 wherein said substitution analog, when aligned with SEQ ID No: 2, shares at least 40% sequence similarity therewith, further wherein said substitution analog shares at least 80% of aligned cysteine residues with said APRIL ligand.

7. Nucleic acid of claim 5 wherein said substitution analog, when aligned with SEQ ID No: 2, shares at least 80% sequence similarity therewith.

8. Nucleic acid having a nucleotide sequence comprising
(a) SEQ ID NO: 1; or
(b) a substitution analog of SEQ ID NO: 1; or
(c) an alteration analog of SEQ ID NO: 1; or
(d) a deletion analog of SEQ ID NO: 1.

9. A vector having the nucleic acid of claim 1, 2, 3, 4, or 8, present as an insert therein, said vector optionally comprising an expression control sequence operably linked to said insert.

10. A host cell comprising the vector of claim 9.

11. A method for producing substantially pure APRIL comprising the steps of:
culturing the host cells of claim 10.

12. An APRIL ligand polypeptide comprising at least about 102 amino acids.

13. An APRIL ligand polypeptide comprising an amino acid sequence found in amino acids 1 to 55 of SEQ ID NO: 2 or a fragment thereof.

14. An APRIL ligand polypeptide comprising an amino acid sequence found in amino acids 157 to 250 of SEQ ID NO: 2 or a fragment thereof.

15. An APRIL ligand selected from:
(a) SEQ ID No: 2; or
(b) an amino acid substitution analog of SEQ D No: 2.

16. A soluble APRIL ligand polypeptide of claims 12-15.

17. A pharmaceutical composition comprising a therapeutically effective amount of an APRIL ligand polypeptide of claims 12-15 and a pharmaceutically acceptable carrier.

19. An antibody that binds specifically to an APRIL ligand polypeptide of claim 12, 13, 14, 15 or 16.

20. An antibody that blocks binding of an APRIL ligand polypeptide to an APRIL receptor polypeptide.

21. An antibody of claim 20 that binds specifically to an APRIL polypeptide.

22. A method for preventing or reducing the severity of an autoimmune disease comprising the step of administering a therapeutically effective amount of a pharmaceutical composition according to claim 17.

23. A method for preventing or reducing the severity of an immune response to a tissue graft comprising the step of administering a therapeutically effective amount of a pharmaceutical composition according to claim 17.

24. A method for stimulating the immune system comprising administering the composition of claim 17.

25. A method for suppressing the immune system comprising administering an effective amount of the pharmaceutical composition according to claim 17.

26. A method for treating cancer comprising administering a therapeutically effective amount of the pharmaceutical composition according to claim 17.

27. A method for identifying a receptor for APRIL comprising:
a. providing APRIL or a fragment thereof,
b. labeling said APRIL or fragment thereof with a detectable label;
c. screening a composition to detect receptors which bind to the detectably labeled of step b.

28. A method of expressing APRIL in a mammalian cell comprising:
a. introducing a gene encoding APRIL into a cell;
b. allowing said cell to live under conditions such that said gene is expressed in said mammal.

29. A method of treating a disorder related to APRIL in a mammal
a. introducing into a cell a therapeutically effective amount of a vector comprising a gene encoding APRIL; and
b. expressing said gene in said mammalian cell.

30. The method of claim 29 wherein the mammal is a human.

31. The method of claim 29 wherein said vector is a virus.

32. A method of inducing cell death comprising the administration of an agent capable of interfering with the binding of APRIL to a receptor.

33. The method of claim 32 further comprising the administration of interferon-K.

34. A method of treating, suppressing, activating or altering an immune response involving a signaling pathway between APRIL and its receptor, said method comprising the step of administering an effective amount of a blocking agent capable of interfering with the association between APRIL and its receptor.

35. The method of claim 34 wherein said immune response involves human carcinoma cells.

36. A method of treating, suppressing or altering the progression of a cancer comprising adminstering to a patient an effective amount of a blocking agent between April and its receptor capable of interfering with the association.

37. The method of claim 36, wherein the blocking agent is a modified inhibitory form of APRIL, or anti-APRIL antibodies or biologically active fragments thereof.

38. The method of claim 37 wherein the blocking agent is an anti-APRIL receptor antibody.

39. The method of claim 36 wherein the blocking agent is administered to a patient in combination with at least one chemotherapeutic agent.

40. The method of claim 39 further comprising the step of administering radiation therapy to said patient.

41. A method of suppressing growth of a tumor cell that expresses APRIL, comprising the step of contacting said cell with an effective amount of an APRIL ligand polypeptide of claims 12-15 or an antibody of claims 19-21.

42. A method of suppressing growth of a tumor cell that expresses an APRIL receptor polypeptide, comprising the step of contacting said cell with an effective amount of a soluble APRIL ligand polypeptide of claims 12-15 or an antibody of claims 19-21 to said subject.
